(19) United States
(12) Patent Application Publication
(10) Pub. No.: US 2005/0147566 A1
(43) Pub. Date: Jul. 7, 2005

(54) Site specific delivery of co-administered drugs via inhalation

(76) Inventors: Scott Fleming, Ewing, NJ (US); Anand V. Gumaste, West Windsor, NJ (US)

Correspondence Address: Norman P Soloway;Haynes Soloway, 130 W Cushing Street, Tucson, AZ, 85701, US

(21) Appl. No.: 10/503,842

(22) PCT Filed: Mar. 12, 2003
(86) PCT No.: PCT/US03/07735

Related U.S. Application Data
(60) Provisional application No. 60/363,585, filed on Mar. 12, 2002.
(60) Provisional application No. 60/417,071, filed on Oct. 9, 2002.

(51) Int. Cl. A61L 9/04; A61K 9/14; A61M 11/00

(52) U.S. Cl. 424/46; 128/200.23

(57) ABSTRACT

Two or more drugs of different particle size are packaged for co-administration to the respiratory pathway.

The present invention relates to the packaging of and co-administration of pharmaceuticals and drugs for medical uses. The invention has particular utility in packaging and administration of precise amounts of two or more pharmaceuticals and drugs to different sites in the respiratory and/or respiratory alimentary pathway, and will be described in connection with such utility, although other utilities are contemplated.

There is a growing trend in the pharmaceutical industry to combine multiple therapeutic medications for the improved treatment of many chronic diseases Examples of this trend are represented in the areas of diabetes and, respiratory and allergy (asthma, COPD) treatment, etc. Specific examples are described below.

New research has demonstrated that the combination of leukotriene receptor antagonists (LTs) with corticosteroids can improve efficacy for asthmatics and with improved safety. LTs are alternatives to long-acting beta-agonists as complementary treatment to inhaled corticosteroids in both pediatric and adult asthma management because they provide bronchodilation and bronchoprotection without development of tolerance, and complement the anti-inflammatory activity unchecked by steroids.

As a result of the above findings, LTs and steroids are currently being co-prescribed with good effect in asthmatics today. The current treatment regimen calls for the patient to take the LT in an oral dose (pill), while the steroid is inhaled using an inhaler. There is currently no product available that delivers these two products in combination. Examples of two LTs on the market are: Merck's Singulair®, chemical name Montelukast, and AstraZeneca's Accolate®, chemical name Zafirlukast. Two highly prescribed corticosteroids on the market are GlaxoSmithKline's Flovent®, chemical name Fluticasone, and AstraZeneca's Pulmicort®, chemical name Budesonide.

The current regimen for the treatment of diabetes as the disease progresses is to use combination therapy to control the blood glucose level in patients. A common practice is to combine an oral dosage medication with injectable insulin. Companies such as GlaxoSmithKline and Eli Lilly have received regulatory approval to market their glitazone products in combination with insulin. These glitazones are currently administered in solid oral dosage forms. There is also considerable work currently underway by companies such as Pfizer-Aventis-Nektar, Novo Nordisk-Aradigm, Eli Lilly-Alkermes, MicroDose Technologies, etc to deliver insulin to the lungs via the inhalation route to treat diabetes.

We believe that by combining the two separate modes of delivery, oral and inhalation, into one mode of delivery i.e. inhalation, will result in higher compliance and therefore improved efficacy.

The present invention provides a medication delivery system in which two or more pharmaceuticals or drugs are delivered to different sites in the respiratory pathway. More particularly, in accordance with the present invention, the particle size of different drugs is controlled according to aerodynamic particle size principles so as to determine the site of action or absorption of the drug in the respiratory pathway. As a result, it is possible to co-administer, simultaneously, by inhalation, two or more different drugs for absorption or depositing in either the mouth or throat where the drug will be dissolved and absorbed in the alimentary canal, and also deliver drugs to the lungs of where the drugs will be absorbed in the respiratory pathway.

As used herein, the term respiratory pathway shall include both the respiratory and alimentary pathways, and shall encompass the nasal and mouth openings, the throat and the lungs.

Further features and advantages of the present invention will be seen from the following detailed description, taken in conjunction with the accompanying drawing, wherein:

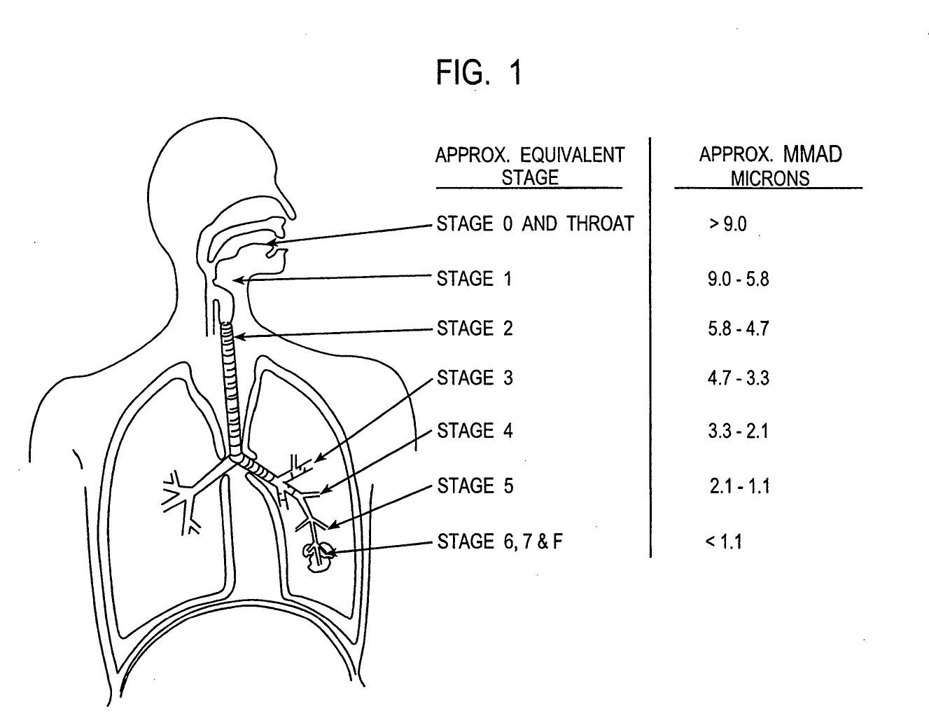

FIG. 1, which is a diagrammatical drawing showing powder dispersion and how it relates to human anatomy.

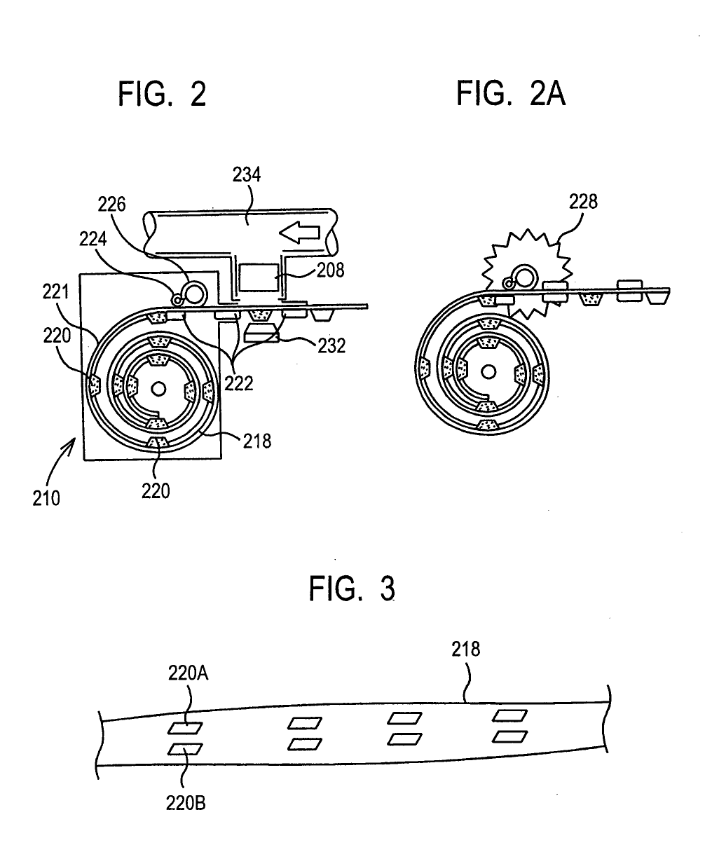

FIG. 2 is a side elevational view of an apparatus made according to the present invention; and

FIG. 3 is a top plan view of a cartridge tape made in accordance with a preferred embodiment of the instant application.

In overview, the present invention is based on the realization that inhaled particles can be delivered to different sites in the respiratory pathway depending upon their aerodynamic particle size. This leads to the ability to direct or control the specific site delivery of pharmaceuticals from an inhaler by tailoring particle sizes. By way of example, and in reference to FIG. 1, dry powder delivered from an inhaler and having a particle size greater than about 9 microns, typically will be deposited in either the mouth or throat where it will dissolve and enter a patient's body through the alimentary canal, and whereas drugs have a particle size less than about 5.8 microns in maximum size will be delivered to the lungs. As seen in FIG. 1, the smaller particle size, the deeper into the lungs will be the delivery.

The present invention provides for co-administration of drug products either simultaneously, sequentially or separately by inhalation. The drugs are delivered to their respective target sites of action, i.e. in the mouth, throat or lungs, through manipulation of the drug particle size. In various embodiments of the invention, the drugs are delivered either from the same drug container simultaneously, i.e. via the same inhalation or puff; simultaneously from separate drug containers; or sequentially from the same or separate drug containers, either in a single inhalation or puff or multiple inhalations or puffs.

In a preferred embodiment of the invention, two or more drugs are delivered simultaneously, i.e. in a single inhalation, using an inhalation device as described in prior U.S. Pat. No. 6,026,809 assigned to the common assignee, but modified to deliver two or more drugs simultaneously. In other words, and with reference to FIG. 2, which corresponds to FIG. 9 of U.S. Pat. No. 6,026,809, the disposable drug cartridge 210 comprises an outer housing 212 which includes a tab 214 for slidably mounting in a recess 216 formed integrally with housing 202. Drug cartridge 210 includes a coiled tape 218 carrying a plurality of spaced bubbles or wells 220 for carrying a dry powder medicament. A release film 221 covers and seals wells 220. Tape 218 is formed as a coil, and is threaded between a first guide platen 222 and pinch roller 224. Pinch roller 224 in turn is driven by a take-up spool 226, which in turn is driven by a thumbwheel 228, which is mounted, on a common shaft with the take-up spool 226. In use, release film 221 is peeled from the tape 218, whereby to expose wells 220, one at a time, as the film is advanced through the cartridge, and the release film 221 is collected on take-up spool 226.

Completing cartridge 210 is a piezoelectric element 232 for mechanically engaging wells 220, as they are selectively advanced in position over and in contact with the piezoelectric element 232. Tape 218 also preferably includes detent means or the like for indexing the tape so that a selected well 220 is automatically positioned over piezoelectric element 232. Finally, an actuating circuit and power supply, similar to that previously discussed, is mounted within cartridge 210.

In one embodiment of the invention, two or more pharmaceuticals or drugs having the same or different particle size may be blended together and loaded in the individual wells 220 for co-delivery. Alternatively, and preferably, as shown in FIG. 3, different pharmaceuticals or drugs having the same or different particle sizes are carried in separate wells 220A, 220B positioned adjacent one another on tape 218 so that the two different pharmaceuticals or drugs may be simultaneously delivered in a single inhalation or puff. Packaging of the different pharmaceuticals or drugs in separate wells 220A, 220B also has the advantage of avoiding possible adverse chemical reaction between the two pharmaceuticals or drugs, reduced formulation demands in terms of homogeneity and settling in blending of the drugs, improved accuracy in filling of the individual drugs into the separate wells and a higher consistency in dose-to-dose repeatability in delivering the drugs.

In another embodiment, the different pharmaceuticals or drugs are loaded in alternate wells 220 along tape 218 whereupon the different pharmaceuticals or drugs may be sequentially administered, i.e. in multiple inhalations.

Summarizing to this point, in accordance with the present invention, particle size of drugs to be administered by inhalation are controlled in order to tailor the delivery of the drug to a selected site in the respiratory pathway or alimentary canal depending on the drug's aerodynamic particle size. This permits selective delivery options including:
1. Buccal delivery—wherein a drug primarily is deposited on buccal mucosa, and the drug has local effect, or absorption takes place through buccal mucosa for systemic effect;
2. Oral delivery—wherein a drug primarily is deposited in the mouth, or throat, and is then swallowed to stomach where it has local effect or is absorbed for systemic effect;
3. Intra-nasal delivery—wherein a drug primarily is deposited in the nasal passages, and has local effect, or is absorbed through the nasal mucosa for systemic effect; and
4. Pulmonary delivery—wherein a drug primarily is deposited on the lungs, and has local effect, or is absorbed through lungs for systemic effect.

The co-administration of separate drug products for inhalation delivery in accordance with the present invention can be grouped into polypharmacy for treatment of a single condition, into polypharmacy for treatment of co-morbid conditions, and for co-administration of separate drug products wherein one drug product is administered to manage side effects resulting from administration of the other drug product. The invention will now be described with reference to the following non-limiting examples:

### Combination I

### A Broncodilator and an Anti-Inflammatory

An Anti-Leukotriene antagonist such as montelukast¹ of particle size about 9 microns for delivery to the mouth for absorption in the alimentary canal, and Budesonide² particles having a particle size of less than about 6 microns for delivery to the lungs.
¹ Montelukast:: [R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid, monosodium salt.

² Budesonide: (RS)-11β,16α,17,21-Tetrahydroxypregna-1,-4-diene-3,20-dione cyclic 16,17-acetal with butyraldehyde.

### Combination II

### Broncodilator and Anti-Inflammatory for Asthma

Budesonide particles as described in Combination I, plus Zafirlukast³ of particle size greater than about 9 microns for delivery to the mouth for absorption in the alimentary canal.
³ Zafirlukast: 4-(5-cyclopentyloxy-carbonylamino-1-methyl-indol-3-ylmethyl)-3-methoxy-n-0-tolylsulfonylbenzamide.

### Combination III

### Oral Agents plus Insulin for Diabetes Management

Insulin of particle size less than about 3 microns for delivery to the lungs, plus a sulfonylurea such as glipizide⁴ of particle size greater than about 9 microns for delivery to the mouth for absorption in the alimentary canal.
⁴ Glipizide: 1-cyclo-hexyl-3-[[p-[2(5-methylpyrazinecarboxamido)-ethyl]-phenyl]sulfonyl]urea.

### Combination IV

### Oral Agent Plus Insulin

Insulin as in Combination III, plus a Thiazolidinedione such as Rosiglitazone maleate⁵ having a particle size greater than 9 microns for delivery to the mouth for absorption in the alimentary canal.
⁵ Rosiglitazone maleate: (+)-5[[4-[2-(methyl-2-pyridinylamino)ethoxyl]phenyl]methyl]-2,4-thiazolidinedi-one, (Z)-2-butenedioate (1:1).

### Combination V

### Oral Agent Plus Insulin

Insulin as in Combination III, plus Acarbose⁶ having a particle size greater than 9 microns for delivery to the mouth for absorption in the alimentary canal.
⁶ Acarbose: O-4,6-dideoxy-4-[[(1S,4R,5S,6S)-4,5,6-trihydroxy-3-(hydroxymethyl)-2-cyclo-hexen-1-yl]amino]-a-D-glucopyranosyl-(1 4)-O-a-D-glu-copyranosyl-(1 4)-D-glucose.

### Combination VI

Insulin as in Combination No. III and a Viguanide such as Metformin⁷ having a particle size greater than 9 microns for delivery to the mouth for absorption in the alimentary canal.
⁷ Metformin: (N,N-dimethylinidodicarbonim-idic diamide hydrochloride).

The following Examples VI and VIII illustrate the co-administration of separate drug products for co-morbid conditions with a high rate of clinic co-occurrence.

### Combination VII

Eighty percent (80%) plus of diabetics are also hypertensive. Therefore, a combination of insulin having a particle size of less than about 3 microns and a drug for controlling hypertension such as Losartan⁸ of a particle size greater than 9 microns for delivery to the mouth for absorption in the alimentary canal.
⁸ Losartan: 2-butyl-4-chloro-1-[p-(o-1H-tetrazol-5-ylphenyl)-benzyl]imidazole-5-methanol monopotassium salt.

### Combination VIII

Insulin of particle size less than about 6 microns for delivery to the lungs, in combination with an ACE Inhibitor such as Lisinopril⁹ of particle size greater than about 9 microns for delivery to the mouth for absorption in the alimentary canal.
⁹ Lisinopril: (S)-1-[N²-(1-carboxy-3-phenylpropyl)-L-lysyl]-L-proline dihydrate.

The following example IX illustrates a combination drug delivery system of the present invention for co-administration of separate drug products where one product is given to manage side-effects (acute or chronic) resulting from the administration of the other drug product.

### Combination IX

Cancer therapies, which include, but are not limited to cytotoxins, often have the side effect of nausea and vomiting. Thus, a combination of a lung cancer therapeutic of particle size less than 6 microns for local or systemic treatment to the lungs, and an anti-emetic of a particle size of greater than 9 microns to delivery to the mouth advantageously may be provided.

It should be noted that other drug combinations might be packaged and delivered in accordance with the present invention without departing from the spirit and scope thereof.

1. A method for delivering drugs via a patient's respiratory pathway which comprises delivering two or more drugs, of different particle size, to different sites in the respiratory pathway.

2. A method according to claim 1, wherein at least one of the drugs is deposited in the mouth or throat for delivery to the alimentary canal.

3. According to claim 1, wherein at least one of the drugs is delivered to the lungs.

4. A method according to claim 2, wherein the drug is delivered to the alimentary canal have a particle size greater than about 9 microns.

5. A method according to claim 3, wherein the particles delivered to the lungs have a particle size less than about 5.8 microns.

6. A method according to claim 1, wherein the two or more drugs are delivered simultaneously.

7. A method according to claim 1, wherein the two or more drugs are delivered sequentially.

8. An inhalation device for delivering drugs to a patient, wherein the drugs comprise two or more drugs having different particle sizes.

9. An inhalation device as claimed in claim 8, wherein at least one of said drugs has a particle size greater than about 9 microns.

10. An inhalation device as claimed in claim 8, wherein at least one of said drugs has a particle size less than about 5.8 microns.

11. An inhalation device as claimed in claim 8, wherein said two or more drugs are packaged together.

12. An inhalation device as claimed in claim 8, wherein said two or more drugs are packaged separately.

13. A method for delivering drugs via a patient's respiratory pathway which comprises delivering two or more drugs of different particle sizes, to different sites in the respiratory pathway where the drugs are from a group of respiratory therapeutic agents.

14. A method according to claim 13 wherein one of the agents is an anti-luketriene antagonist.

15. A method according to claim 13 wherein one of the agents is a cortico-steroid.

16. A method for delivering drugs via a patient's respiratory pathway which comprises delivering two or more drugs of different particle sizes, to different sites in the respiratory pathway where the drugs are from a group of diabetic control agents.

17. A method according to claim 16 wherein one of the drugs is insulin.

18. A method according to claim 16 wherein one of the drugs is an oral agent such as glipizide and/or thiazolidinedione and/or acarbose and/or viguanide.

19. A method for delivering drugs via a patient's respiratory pathway which comprises delivering two or more drugs of different particle sizes, to different sites in the respiratory pathway where the drugs represent a combination of drugs to treat the co-morbid condition of diabetes and/or hyperlipidemia and/or hypertension.

20. A method according to claim 19 wherein one of the drugs comprises a statin.

21. A method according to claim 20 wherein one of the statins comprises Lovastatin or Simvastatin.

22. A method according to claim 19 wherein one of the drugs is selected from the group consisting of one or more of an ACE inhibitor, a calcium channel blocker, and an ARB.

23. A method for delivering drugs via a patient's respiratory pathway which comprises delivering two or more drugs of different particle sizes, to different sites in the respiratory pathway where one of the drugs is given to treat or manage the side effects of the other drug.

24. An inhalation device for delivering drugs to a patient, wherein the drugs comprise two or more drugs having different particle sizes and where the drugs are from a group of respiratory therapeutic agents.

25. A method according to claim 24 wherein one of the agents is an anti-luketriene antagonist.

26. A method according to claim 24 wherein one of the agents is a cortico-steroid.

27. An inhalation device for delivering drugs to a patient, wherein the drugs comprise two or more drugs having different particle sizes and where the drugs are from a group of diabetic control agents.

28. A method according to claim 27 wherein one of the drugs is insulin.

29. A method according to claim 27 wherein one of the drugs is selected from the group consisting of one or more of glipizide, thiazolidinedione, acarbose and viguanide.

30. An inhalation device for delivering drugs to a patient, wherein the drugs comprise two or more drugs having different particle sizes and where the drugs represent a combination of drugs to treat the co-morbid of diabetes and/or hyperlipidimia and/or hypertension.

31. A method according to claim 30 wherein one of the drugs comprises a statin.

32. A method according to claim 31, wherein one of the statins comprises Lovastatin or Simvastatin.

33. A method according to claim 30 wherein one of the drugs is selected from the group consisting of one or more of an ACE inhibitor, a calcium channel blocker, and an ARB.

34. An inhalation device for delivering drugs to a patient, wherein the drugs comprise two or more drugs having different particle sizes and where one of the drugs is given to treat or manage the side effects of the other drug.
